(19) United States
(12) Patent Application Publication
(10) Pub. No.: US 2009/0124893 A1
(43) Pub. Date: May 14, 2009

(54) APPARATUS AND METHOD TO PERFORM STEREOTACTIC TREATMENT OF MOVING TARGETS

(76) Inventor: Alexander Schlaefer, Fuerstenberg (DE)

Correspondence Address: Alexander Schlaefer, Wallstrasse 14, Fuerstenberg, 16798, omitted

(21) Appl. No.: 12/271,276

(22) Filed: Nov. 14, 2008

Related U.S. Application Data
(60) Provisional application No. 60/988,087, filed on Nov. 14, 2007.

(51) Int. Cl. A61B 5/05

(52) U.S. Cl. 600/426

(57) ABSTRACT

In accordance with an embodiment, an apparatus determines the location of landmarks inside a patient. The apparatus includes imaging units containing two-dimensional imaging arrays capable of emitting and receiving signals to obtain three-dimensional images. The imaging units are attached to the patient's body and their location is established with a tracking system and used to compute the position of landmarks from the images. The images are adjusted relative to the imaging units such that the landmarks remain inside the images during motion of landmarks or imaging unit. The apparatus also includes a control and computing device to process the image data and to adjust the imaging arrays accordingly. In accordance with one embodiment, sources of treatment are directed towards a target structure in the patient's body.

This application claims the benefit of U.S. Provisional Application No. 60/988,087 filed on Nov. 14, 2007.

### FIELD OF THE INVENTION

The invention relates to determining the location of a lesion or anatomical structure during medical procedures. In particular, the invention relates to obtaining a set of images and their respective position, to using this set of images to establish the position of anatomical or artificial landmarks, and to direct treatment accordingly.

### BACKGROUND OF THE INVENTION

Medical procedures for treatment of internal lesions or tumors in a patient often require precise knowledge of the location of the respective target region. Examples include external beam radiation therapy or highly focused ultrasound, where spatially exact treatment is performed solely from outside the patient.

In external beam radiation therapy, a beam of ionizing radiation is used for treatment of tumors inside the patient. The combined dose delivered by a set of beams starting at different positions and oriented towards the tumor is chosen such that the tumor is destroyed and healthy tissue surrounding the tumor is spared as much as possible. To find a set of suitable beams the tumor is located based on an image of the patient before treatment starts. Typically, computed tomography (CT) and magnetic resonance imaging (MRI) are used for this purpose.

However, many lesions or tumors are subject to spontaneous or systematic motion during treatment. To handle such motion, e.g., by redirecting the beam appropriately, it is necessary to determine the location of the target region throughout treatment. Different approaches to accomplish target tracking have been proposed.

A method described by Adler in U.S. Pat. No. 5,207,223 is based on small artificial landmarks implanted in close proximity to the target. The landmarks have a high density compared to anatomical tissue and can be easily identified in x-ray images. By taking images with two x-ray camera systems simultaneously it is possible to compute the position of the artificial landmarks. Yet, due to the extensive x-ray exposure to the patient images cannot be obtained continuously. Therefore, the method has been extended to handle systematic motion by correlating the landmark location computed from the x-ray images with an external respiratory signal. When the correlation model has been established the external respiratory signal alone is sufficient to infer the landmark position. In U.S. Pat. No. 6,144,875 Schweikard and Adler propose to use an optical tracking system and markers on the patient's skin as external signal source.

One limitation of the method given by Adler and Schweikard is the necessity to implant artificial landmarks. In U.S. Pat. No. 7,260,426 Schweikard and Adler describe a method based on natural landmarks, e.g., bony structures, in the proximity of the tumor. While this approach has been successfully tested with tumors in the thorax, it is of limited use in the lower abdomen, where no moving bony structures are present. Another limitation lies in the correlation model, which is based on the assumption of systematic or cyclic motion. While organs in the abdomen are subject to systematic motion, e.g., respiratory motion, there is also substantial spontaneous motion, specifically in the lower abdomen.

Another method for tumor tracking is based on locating an implanted marker directly. The system described in U.S. Pat. No. 20,020,193,685 uses excitable markers that generate radio-frequency magnetic signals measurable from outside the body. Limitations of this approach include the relatively low measurement frequency due to the need to excite the marker and subsequently read its position, the invasiveness of the marker implantation, and particularly the need for a suitable treatment environment that does not affect the measurement of the signals or otherwise distort the magnetic field.

It would be desirable to overcome the aforementioned limitations. Specifically, a system for target tracking should measure the actual target position regardless of the type of motion. Furthermore, the system needs to deliver real-time position information and should not expose the patient to x-ray radiation. It also needs to compatible with the treatment method, specifically it needs to be small enough to avoid interference or collision with the treatment device. Moreover, such a system should allow for completely non-invasive target tracking of anatomical landmarks.

It would also be desirable to use the information on the target position and motion to adjust sources of therapeutic treatment accordingly. Specifically, this includes but is not limited to a system where multiple sources of ultrasound or microwave radiation are oriented such that a common target region is covered throughout treatment.

### SUMMARY OF THE INVENTION

The present invention is directed toward improved continuous localization of landmarks inside a patient. It can used to determine the motion of a clinical target structure and to direct the treatment accordingly.

In accordance with one embodiment of the invention, an apparatus determines the position of landmarks inside the patient's body. The apparatus contains one or more two-dimensional imaging arrays of signal emitters and receivers, where the signal is in the ultrasound or microwave range, and where the sent and received signals are processed to obtain a three-dimensional image. In accordance with the invention, each two-dimensional imaging array is part of an imaging unit, where the orientation of the two-dimensional imaging array within that unit can be rotated about both image axes. Each imaging unit is attached to the patient's body. Methods to attach the units include but are not limited to adhesive pads and belts or vests. Furthermore, each imaging unit is connected to a central control unit, and the position and orientation of each imaging unit is tracked by a localization system. Therefore, the three-dimensional images obtained by the different imaging units can be mapped into one global three-dimensional coordinate frame. Hence, it is possible to identify the same landmark in a number of the three-dimensional images. Using the position of the landmark in the images, the position and orientation of the respective imaging units as measured by the localization system, and the orientation of the two-dimensional array within each unit the central control unit computes the landmark position in the global coordinate frame. Landmarks according to the invention could be either anatomical structures well visible in the images, for example vessel bifurcations, or artificial landmarks implanted inside or in close proximity of the target region. Initially, the relative position of landmark and target region will be defined by the operator, for example based on images of the patient, including but not limited to CT and MRI. Throughout treatment the spatial relationship between landmark and target region will allow for localization of the target region when the landmark position is established. Moreover, artificial landmarks can be made of special material, for example material that strongly reflects the signals, such that the landmarks can be easily detected in the images.

In accordance with another embodiment of the invention the force of the imaging unit pushing against the patient body can be adjusted to control the image quality.

In accordance with another embodiment the apparatus may comprise a treatment device which can move and redirect the treatment. Such a device may consist of a moveable source of ionizing radiation, where the beams of radiation are directed according to the position of landmark and target region.

In another embodiment a treatment device may consist of multiple sources of ultrasound or microwave radiation in a frequency range and intensity suitable for therapeutic use. These sources are part of treatment units, where each treatment unit is attached to the patient and connected to the control unit, and the position and orientation of each treatment unit is measured by a localization system and mapped into the same global coordinate frame as the images and landmark positions. The sources of ultrasound or microwave radiation may be rotated with respect to the embedding treatment unit with at least two degrees of freedom, such that any changes in the relative position between the treatment unit and the landmark are compensated by rotating the source accordingly. Therefore, in accordance with the invention, the control unit computes the landmark position as detailed before, and furthermore processes the position and location of the treatment units, and controls the orientation of the sources of ultrasound or microwave radiation such that the target region is properly covered throughout treatment.

### BRIEF DESCRIPTION OF THE DRAWINGS

FIG. 1 is a diagram illustrating a two-dimensional axial view of a landmark localization scenario in accordance with the invention;

FIG. 2 is a diagram illustrating a two-dimensional sagittal view of a landmark localization scenario in accordance with the invention;

FIG. 3 is a two-dimensional illustration of one possible embodiment of imaging unit and imaging array in accordance with the invention;

FIG. 4 illustrates how the imaging array may be rotated about two axes in one possible embodiment in accordance with the invention;

FIG. 5 is a two-dimensional diagram illustrating an example where a landmark position is detected by an imaging unit in one embodiment in accordance with the invention;

FIG. 6 is a two-dimensional diagram illustrating the situation when the imaging array orientation has been adjusted according to the detected landmark position;

FIG. 7 is a block diagram showing system components of one embodiment in accordance with the invention;

FIG. 8 illustrates the computation of the landmark position from the orientation and position of different imaging units and imaging arrays in accordance with one embodiment of the invention;

FIG. 9 illustrates how adjustable treatment sources attached to the patient can be used to perform stereotactic treatment in accordance with one embodiment of the invention;

### DETAILED DESCRIPTION OF A PREFERRED EMBODIMENT

In the following description, certain specific details are set forth in the context of radiation therapy, as this provides a well know environment to discuss various embodiments of the invention. However, one skilled in the relevant art will recognize that the invention may be practiced without one or more of these specific details, or with other methods, components, materials, etc. Particularly, the description discusses further embodiments, where the treatment is delivered by ultrasound or microwave radiation with a frequency and intensity suitable for therapeutic use. Moreover, the following discussion will consider ultrasound images for localization of the target region. It will be appreciated that a wider range of image modalities, including but not limited to microwave imaging, can be similarly used in accordance with the invention.

In an initial step before treatment starts, the target region and suitable landmarks need to be defined. For radiation therapy, CT or MRI are typically used for this purpose. In the context of the invention, the imaging units used for localization during treatment may also be used to identify the target region and the landmarks. Landmarks are chosen such that they are inside or in close proximity of the target region and easily detected in the images provided by the imaging units. Examples for natural landmarks include vessel bifurcations with a distinctive size and shape, or other distinctive image features related to some anatomical structure. Where no suitable natural landmarks are available artificial landmarks may be implanted inside or close to the target region. These landmarks may be made of a special material such that the landmarks are easily detectable with the imaging units. For sake of clarity we continue the description of an embodiment of the invention for one landmark, but it is recognized that multiple landmarks may be used.

In one embodiment, imaging units are attached to the patient and controlled such that the landmark is always visible in the respective image. Examples for methods to attach the imaging units to the patient include but are not limited to the use of a belt or a vest, or adhesive pads. Examples for methods to control the direction of the image are mechanical rotation of the image array about at least two independent axes, or a delay of the sent and received signals known as beam steering. When ultrasound is used the imaging unit is made of ultrasound compatible material and may feature a deformable underside that adjusts to the patient's skin surface. In accordance with embodiments of the present invention the force pushing parts of the underside of the imaging unit against the patient surface may be adjusted, for example using electric or piezo-electric actuators, or by injecting ultrasound gel between imaging unit and patient surface.

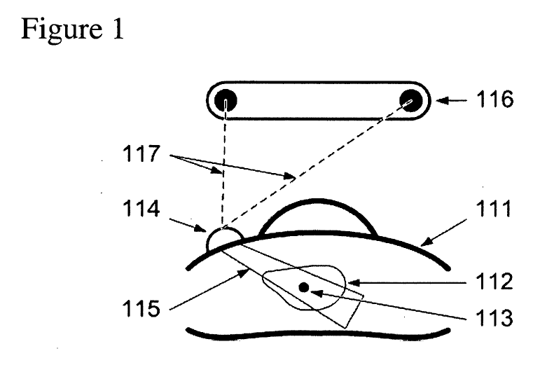

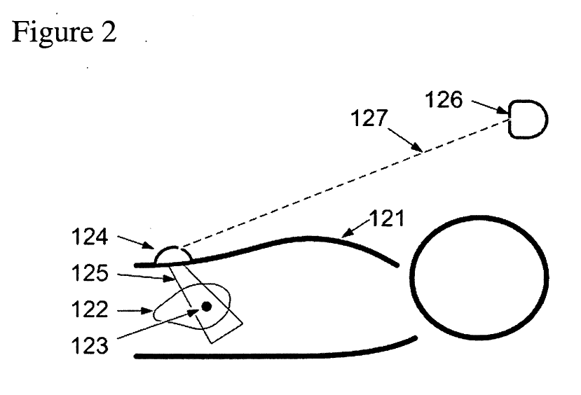

FIGS. 1 and 2 illustrate the landmark localization for an axial and a sagittal view of the patient. In FIG. 1, the imaging unit 114 is attached to the right side of the abdominal wall of the patient 111. The image 115 is covering the landmark 113, which itself is inside the target region 112. Furthermore, the position of the imaging unit 114 is tracked by the localization system 116 through signals 117. FIG. 2 depicts the same situation in a sagittal view, where the imaging unit 124 is attached to the patient's abdominal wall 121, and the imange 125 covers the landmark 123 inside the target region 122. The position of the imaging unit 124 is tracked by the localization system 126 through signals 127.

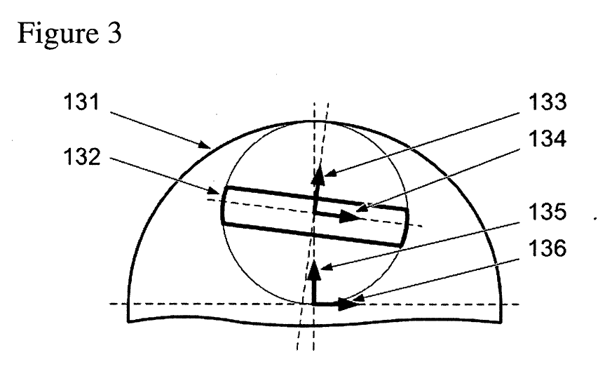

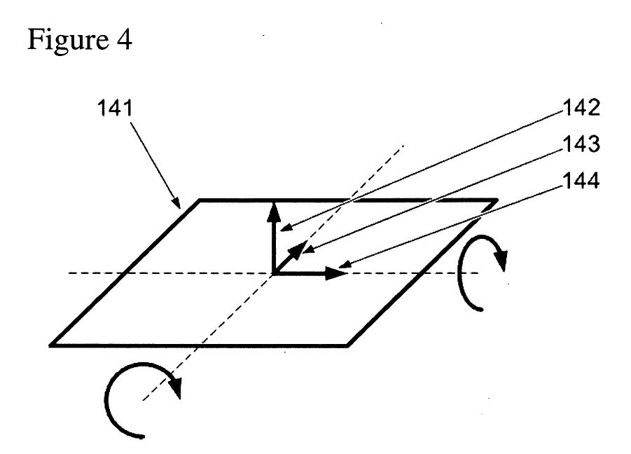

In one embodiment, each imaging unit contains a two-dimensional imaging array and is made of a material compatible with the imaging modality. FIG. 3 shows a two-dimensional view of an imaging unit 131 containing an imaging array 132. The figure shows the x-axis 134 and the z-axis 133 of the x-y-z coordinate system related to the imaging array, and the x-axis 136 and the z-axis 135 of the x-y-z coordinate system related to the imaging unit. Note that in the figure the imaging array is rotated around its y-axis. In general, rotation is possible around at least two of the three imaging array coordinate system axes. FIG. 4 illustrates one embodiment in accordance with the invention where the two-dimensional imaging array 141 can be rotated around the x-axis 144 and the y-axis 143 of the related x-y-z coordinate system, where 142 is the z-axis. A plurality of mechanical systems may be used to achieve the desired rotations.

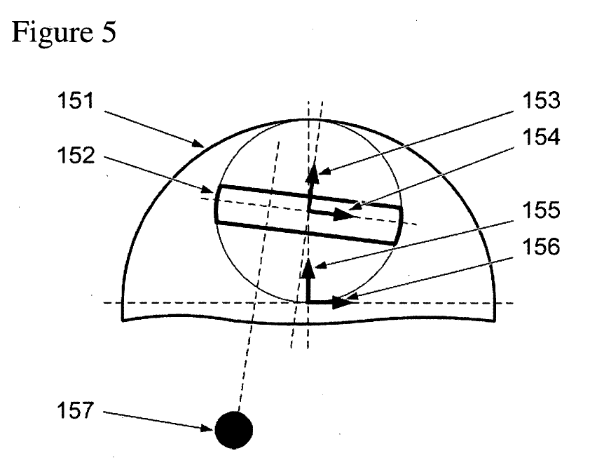

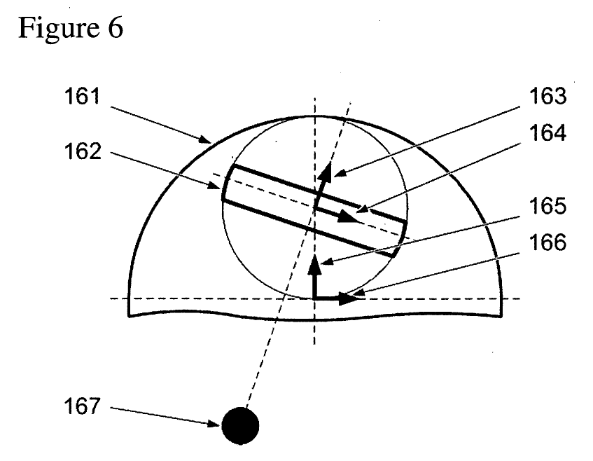

In one embodiment, the actual image is obtained by emitting a signal and measuring various properties of the signal reflected by the tissue and material inside the patient, for example as done in three-dimensional ultrasound systems. The imaging array used in accordance with the invention may be small, and the array may be rotated to track the landmark. FIG. 5 illustrates a situation where the landmark 157 detected in the image does not lie on the z-axis 153 of the imaging array coordinate system. The figure also shows the x-axis 154 of the imaging array coordinate system and the z-axis 155 and x-axis 156 of the imaging unit coordinate system to highlight the rotation between imaging unit 151 and imaging array 152. When the image has been processed by the control unit, the rotation of the imaging array is adjusted such that the expected position of the landmark lies on the z-axis of the imaging array coordinate system. The resulting situation is depicted in FIG. 6, showing the imaging unit 161, the imaging array 162, the x-axis 164 and z-axis 163 of the imaging array coordinate system and the x-axis 166 and z-axis 165 of the imaging unit coordinate system, and the landmark 167. Thus, the imaging unit can track a moving landmark as long as the landmark is visible in two consecutive images. The rotation of the image in accordance with the invention may be achieved by mechanical rotation of the image array, or by beam steering, or by a combination of both.

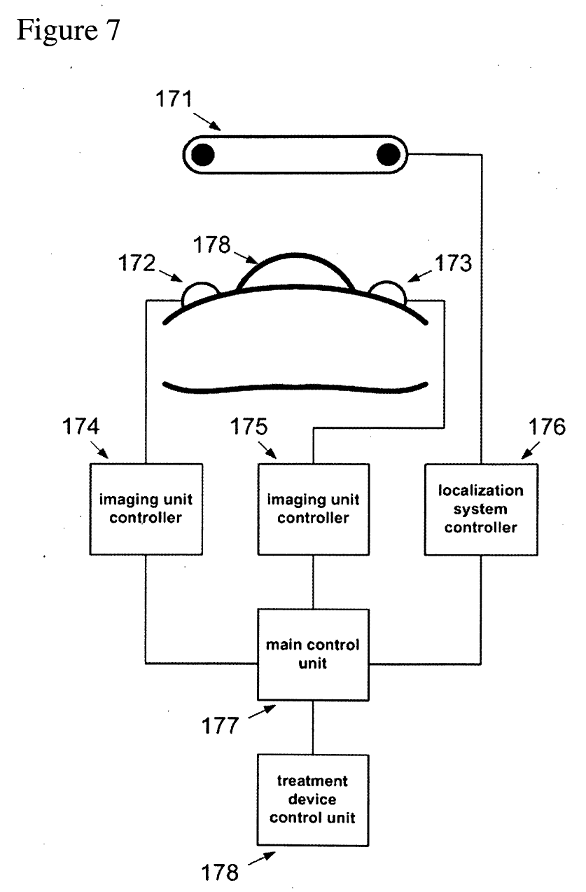

FIG. 7 shows a block diagram with components of an embodiment in accordance with the invention. Two imaging units 172 and 173 are attached to the patient 178 and tracked by a localization system 171. The localization system is connected to the localization system controller 176, which processes the position and orientation data. Each imaging units is connected to a respective imaging unit controller 174 and 175, which performs basic image processing and controls the rotation of the imaging arrays accordingly. The main control unit 177 further processes the position data and instructs the treatment device control unit 179 to adjust the treatment accordingly. All controllers may also be implemented in a single piece of hardware.

In one embodiment, orientation and position of the image with respect to the imaging unit can be directly derived from the rotation of the imaging array and the parameter for beam steering. The imaging unit controller determines the position of the landmark in the image to compute a line with respect to the imaging unit coordinate system, such that this line is parallel to the z-axis of the imaging array and runs through the center of the landmark. In one aspect, the landmark position can be computed from this line, the distance between landmark and imaging array as determined in the image, and the position of the imaging unit with respect to the localization system. In another aspect, the landmark position is computed as the intersection point of lines determined by at least two imaging units. In yet another aspect, multiple lines from multiple imaging units and the respective distances between landmark and imaging units are used to compute the most likely landmark position.

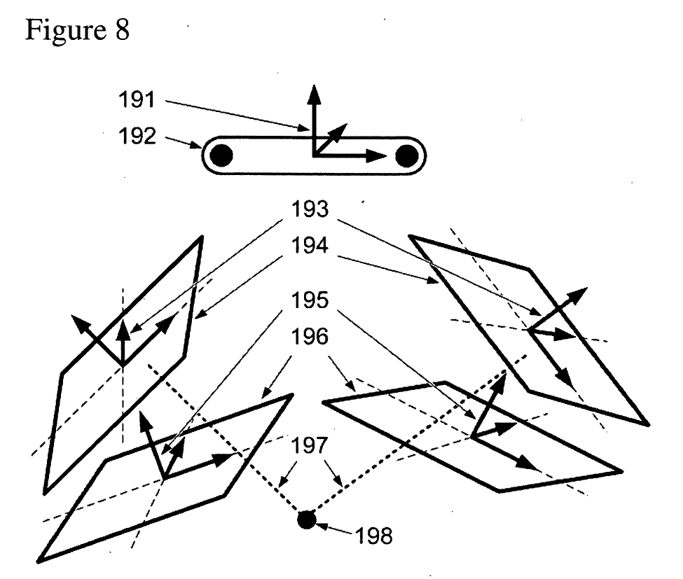

FIG. 8 illustrates the concept for two imaging units, represented by their bases 196 and the related coordinate systems 195. For the imaging arrays 194 the rotation and position of the related coordinate systems 193 with respect to the imaging unit coordinate system is known. The landmark 198 is detected in both images, leading to the lines 197. Using the known relationship between imaging array coordinate systems and imaging unit coordinate system, and the fact that the position and orientation of both imaging units is tracked with the localization system 192, both lines can be expressed with respect to the localization system coordinate system 191. The landmark position may be computed as the intersection of the two lines.

In one embodiment of the invention, movable imaging arrays inside imaging units attached to the patient are used to track a landmark position. In another aspect of the invention a similar principle is applied to treatment devices. Hence, the illustrations given in FIGS. 1-6 apply likewise to the case where one or more treatment units are attached to the patient. The actual source of treatment is mounted inside the treatment unit and can be rotated around at least two axes. Given the known position of the target region, and the position and orientation of the treatment unit as tracked by the localization system, the rotation of the treatment source is controlled such that the treatment covers the target region or a specified part thereof. Other parameters of the treatment, including but not limited to the treatment focus, may also be adjusted according to the relative position of treatment unit with respect to the target area. Potential treatment modalities include but are not limited to ultrasound and microwave radiation. As such, the system uses the information on the position of the target region to adjust the treatment accordingly. It will be appreciated that the information on the position of the target region can be obtained using a plurality of systems, including but not limited to the system outlined in the previous sections.

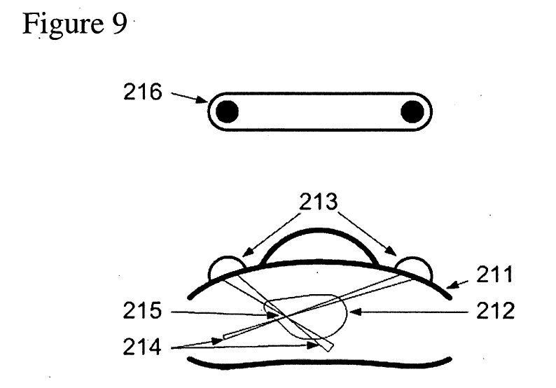

The treatment aspect of the invention is illustrated in the context of focused ultrasound. FIG. 9 shows two treatment units 213 attached to the patient 211. The focussed ultrasound treatment 214 is directed to a focus point 215 inside the target region 212. The position and orientation of the treatment units is tracked by the localization system 216, and the parameters of the treatment are adjusted such that the same point relative to the target region is targeted for the planned amount of time.

In one embodiment, using the treatment parameters for each treatment unit may be specified such that potentially harmful effects to tissue passed by the treatment are limited while the cumulative effect from all treatment units inside the target region is at a therapeutic level. Therefore, one advantage of the invention lies in the fact that it enables more precisely targeted treatment.

Since certain changes may be made in the above devices and processes without departing from the scope of the invention described herein, it is intended that all matter contained in the above description or shown in the accompanying drawings shall be interpreted in an illustrative and not in a limiting sense.

1. An apparatus and method to measure and monitor the position of one or more landmarks inside a patient, comprising: one or more imaging units, where each imaging unit contains a two-dimensional imaging array capable of emitting and receiving signals to obtain three-dimensional images, and where the orientation of the image with respect to the imaging unit can be adjusted; a localization system to track the position and orientation of the imaging units; a control unit connected to the imaging units and the localization system; and steps to detect the landmarks in the images, to compute the landmark position from the images and the information on the orientation and position of the imaging units and the orientation of the image; and to control the imaging array such that the landmark remains in the respective images.

2. The apparatus and method of claim 1, where the landmark is an anatomical structure showing distinctive image features.

3. The apparatus and method of claim 1, where the landmark is implanted to the patient and made of material easily detectable in the images.

4. The apparatus and method of claim 1, where the imaging units are attached to the patient using a belt, vest, or adhesive pad.

5. The apparatus and method of claim 1, where the signals are ultrasound signals.

6. The apparatus and method of claim 1, where the signals are in the microwave range.

7. The apparatus and method of claim 1, where the orientation of the image with respect to the imaging unit is adjusted by rotation of the imaging array about at least two independent axes.

8. The apparatus and method of claim 1, where the orientation of the image with respect to the imaging unit is adjusted by beam steering.

9. The apparatus and method of claim 1, where the resulting position data is used to adjust a treatment device to point towards a moving target.

10. The apparatus and method of claim 9 where the treatment device is a radiation therapy system.

11. The apparatus and method of claim 9 where the treatment device is a highly focused ultrasound system.

12. An apparatus and method to perform stereotactic treatment with treatment sources attached to a patient, comprising: on or more treatment units, where each treatment unit contains a treatment source, and where the orientation of the treatment with respect to the treatment unit can be adjusted; a localization system to track the position and orientation of the treatment units; a system to detect and track the position of the treatment target region; a control unit connected to the treatment units, the target region tracking system, and the localization system; and steps to compute the treatment parameters and adjust the treatment such that the desired region within the target region is treated regardless of the motion of target region and treatment unit.

13. The apparatus and method of claim 12, where the treatment units are attached to the patient using a belt, vest, or adhesive pad.

14. The apparatus and method of claim 12, where the treatment sources emit microwave radiation or ultrasound in a frequency and intensity suitable for therapy.

15. The apparatus and method of claim 14, where highly focused ultrasound is used.

16. The apparatus and method of claim 14, where the focal point can be adjusted.

17. The apparatus and method of claim 12, where the orientation of the treatment with respect to the treatment unit is adjusted by rotation of the treatment source about at least two independent axes.

18. The apparatus and method of claim 12, where the treatment source comprises a two dimensional arrangement of emitters.

19. The apparatus and method of claim 18, where the orientation of the treatment with respect to the treatment unit is adjusted by beam steering.
